SEQUENCE LISTING (3 sequences)

SEQ ID NO: 1 - DNA, 1103 bp, Heterodera glycines
ggtttaattacccaagtttgaggcatttgttcactttaatattttgccatgtcttttcttgtcacattagccggctatttgacaattgccatttttctccagaaactctttcgctctctgtacaatgtggttttcccctatttgttcgctgttcctcagaatatcaaggtgctcgcgggtgccgagtgggcagtggtcaccggcagcactgacggcattggcaaggcgtacgcgctcgagttggccaagaaaaacttcaacgtggtgctcatctcacgctccatggacaaactgaacgcagtggcggaggaaatcaaacagaaatatcccaatgtggaagtgaagtgcatctcatttgacttcaccaacgcaaatctgaaggactacgagcagacaattttctcccaactttccaccatcgaagttggaatgctcgtgaacaatgtcgggatgagctacgagtaccccgagcgattggaccgtattgagggcggactgcaacgcgtctccgacattaccgtgatcaacaccctgccgactacggtactctccgcttttgtgctcaaacagatgagggaacggggacgcggcgtcgtcgtcaatttggcctcctcggcagcgtatttcaattggttttacctggccgcttattccgcgtcgaagaaatacgtgtgttggttgtcctccattctgcgcatggaatacgccgacaccgacattgtcatccaaacggtgtgcccgatgatggtggccaccaaaatggccaagattcgcaaggcttccttcttcacgcccagccccgaggaatttgccgcccaagcgctccgcagcattggcttggtcgacgagaccaccggctgtttgtcccaccaaattcaggcggagctgatgttcggctacgtccccgccccgttgctgaacaaattcgtgcgcgacaactcactgcagacgcgcaagcgcgccctgaagaagaaggaagcgacggtggcgaagagtgaatgatgagaaaaaagaaagaaaagtgatgtgaaatattgacattcattgaaacaaccaaagtaaattaaagttatttaataaccaaaaaaaaaaaaaaaaaaaaa

SEQ ID NO: 2 - DNA, 719 bp, Heterodera glycines
ggtttaattacccaagtttgagtacgattattatcttgcacgaatccaattgtttcctatcttagacacttaaatggcactgaaacgtatacaaaaggagctgcaggacctcggccgtgacccacccgcccagtgcagtgctggccctgtcggcgatgaccttttccattggcaagccaccattatggggccaccagaatcgccttaccagggcggcgtcttctttctgaccatccacttcccgacagactatccgtttaagccaccgaaggtggcgtccaccactcgtatttatcatccgaacatcaacagcaacgggagcatttgccttgatattctgagatctcaatggtctcctgcactgactatctccaaagtcttgctttcgatttgctctcttctctgtgatccgaatcccgatgatccattggttccggagatagcacgcatctacaagacggatcgcgaaagatacaatacgttggcgcgggaatggactcagaaatatgcgatgtgatcgacggacattgccacccgggaagaataccctcacgaccatcccttacatccaccactgaattttcttttggagtcttctctattgtgaaagcgaactttttggaaaatcggcctctcctttattatgtcgcccaagacaaatgtttttcggtaacaagtaaataaatggttgaaacacaaaaaaaaaaaaaaaaaaaaaa

SEQ ID NO: 3 - DNA, 1325 bp, Heterodera glycines
ggtttaattacccaagtttgagataataggaatcctttctatcattattcaaaatgtctcaccgtaaattttctgcacctcgccacgggtccatgggcttcacgcccaagaagcgagcgaagcgtcatcgcggaaaggtcaaggctttcccgaaggatgacccaaagaaacccatccatctgacggcgttcatcgcctacaaggctggcatgtcacatattgtgcgcgacgtggacaaacttggctcgaaggtgcacaaaaaggaagtggtggaggcggtcacaattctcgagacgcctcccatggtcattgtcggtgtggtcggttacatcgaaacgccgcgcgggccgcgtccgctcaaaaccgtttttgctgagcatttgagcgaggactgcaaaagacgtttctacaagaattggtacaagtgcaagaagaaggcattcacgaagtatgccaagaaatggcaagacgaggcgggacagaaatccatcgaggccgacctgaataaaattaagaagtactgttcttatgtgcgcgtcattgctcacacccagatgaaactgatgaagcaccgcaacaagaaggcacacataatggagatccaaatcaatgggggtaccgttgccgaaaaagtggactgggctcgtgagcactttgagaagcaggttcccgtggagcaagtgtttgagcaggacgagatgatcgactgcatcggtgtcaccagaggaaagggtttcaagggagtgaccagccgatggcactgtaagaagttgccgcgcaagactcacaagggtctccgcaaggtggcttgtatcggtgcatggcatccgtcgcgtgtctcattcacagttgcgcgtgccggtcagaagggctaccaccatcggacggagatcaacaagaagatctaccgcattggcaagtcgtgtttgacggccgacggcaagaaaaacggaatgaccgacttcgacatcactgagaagaacattaacccgatgggcgggttcccccactatggtctggtcaatcaggactttctgatgatccgtggctgttgcattggttgcaagaagcgtccgatcacattgcgcaagactttgatcaagcagaccaaacgttttgctttcgagaagataaacctgaagttcatcgacaccagctccaagtttggccatggacgcttccagacgagtgcggagaagaaaacattcatgggcaaattgaagaaagactttctggcggaatcggcgtaaaattggccaatttcattgtttattattttgttgaaagtaaagaattgttgtgttaaaaaaaaaaaaaaaaaaaaa

(19) United States
(12) Patent Application Publication
(10) Pub. No.: US 2008/0311555 A1
(43) Pub. Date: Dec. 18, 2008

(54) In Vitro Root-Nematode Assay in Multi-Well Plates

(75) Inventor: Xiang Huang, Apex, NC (US)

Correspondence Address: BASF CORPORATION, CARL-BOSCH-STRASSE 38, LUDWIGSHAFEN, D67056, DE

(73) Assignee: BASF Plant Science GmbH, Ludwigshafen (DE)

(21) Appl. No.: 12/132,727

(22) Filed: Jun. 4, 2008

Related U.S. Application Data
(60) Provisional application No. 60/944,181, filed on Jun. 15, 2007.

(51) Int. Cl. C12Q 1/00

(52) U.S. Cl. 435/4

(57) ABSTRACT

The invention provides an improvement in the method of screening roots, both transgenic and non-transgenic, for activity against parasitic nematodes. In particular, the improvement of the invention is use of multi-well cell culture plates for root culture and nematode infection steps.

### CROSS REFERENCE TO RELATED APPLICATIONS

This application claims priority benefit of U.S. provisional patent application Ser. No. 60/944,181, filed Jun. 15, 2007.

### BACKGROUND OF THE INVENTION

1. Field of the Invention

The field of this invention is the control of nematodes, in particular the control of plant parasitic nematodes. The invention also relates to assays of genetic material in plants to determine whether such genetic material increases plant resistance to nematodes.

2. Background Art

Nematodes are microscopic wormlike animals that feed on the roots, leaves, and stems of more than 2,000 vegetables, fruits, and ornamental plants, causing an estimated $100 billion crop loss worldwide. One common type of nematode is the root-knot nematode (RKN), whose feeding causes the characteristic galls on roots. Other root-feeding nematodes are the cyst- and lesion-types, which are more host specific.

Nematodes are present throughout the United States, but are mostly a problem in warm, humid areas of the South and West, and in sandy soils. Soybean cyst nematode (SCN), Heterodera glycines, was first discovered in the United States in North Carolina in 1954. It is the most serious pest of soybean plants. Some areas are so heavily infested by SCN that soybean production is no longer economically possible without control measures. Although soybean is the major economic crop attacked by SCN, SCN parasitizes some fifty hosts in total, including field crops, vegetables, ornamentals, and weeds.

Signs of nematode damage include stunting and yellowing of leaves, and wilting of the plants during hot periods. However, nematodes, including SCN, can cause significant yield loss without obvious above-ground symptoms. In addition, roots infected with nematodes are dwarfed or stunted. Nematode infestation can decrease the number of nitrogen-fixing nodules on the roots, and may make the roots more susceptible to attacks by other soil-borne plant pathogens.

The nematode life cycle has three major stages: egg, juvenile, and adult. The life cycle varies between species of nematodes. For example, the SCN life cycle can usually be completed in 24 to 30 days under optimum conditions where other species can take as long as a year or longer to complete the life cycle. When temperature and moisture levels become adequate in the spring, worm-shaped juveniles hatch from eggs in the soil. These juveniles are the only life stage of the nematode that can infect soybean roots.

The life cycle of SCN has been the subject of many studies and therefore can be used as an example for understanding a nematode life cycle. After penetrating the soybean roots, SCN juveniles move through the root until they contact vascular tissue, where they stop and start to feed. The nematode injects secretions that modify certain root cells and transform them into specialized feeding sites. The root cells are morphologically transformed into large multinucleate syncytia, which are used as a source of nutrients for the nematodes. The actively feeding nematodes thus steal essential nutrients from the plant resulting in yield loss. As the nematodes feed, they swell and eventually female nematodes become so large that they break through the root tissue and are exposed on the surface of the root.

Male SCN nematodes, which are not swollen as adults, migrate out of the root into the soil and fertilize the lemon-shaped adult females. The males then die, while the females remain attached to the root system and continue to feed. The eggs in the swollen females begin developing, initially in a mass or egg sac outside the body, then later within the body cavity. Eventually the entire body cavity of the adult female is filled with eggs, and the female nematode dies. It is the egg-filled body of the dead female that is referred to as the cyst. Cysts eventually dislodge and are found free in the soil. The walls of the cyst become very tough, providing excellent protection for the approximately 200 to 400 eggs contained within. SCN eggs survive within the cyst until proper hatching conditions occur. Although many of the eggs may hatch within the first year, many also will survive within the cysts for several years.

Nematodes can move through the soil only a few inches per year on its own power. However, nematode infestation can be spread substantial distances in a variety of ways. Anything that can move infested soil is capable of spreading the infestation, including farm machinery, vehicles and tools, wind, water, animals, and farm workers. Seed sized particles of soil often contaminate harvested seed. Consequently, nematode infestation can be spread when seed from infested fields is planted in non-infested fields. There is even evidence that certain nematode species can be spread by birds. Only some of these causes can be prevented.

Traditional practices for managing nematode infestation include: maintaining proper soil nutrients and soil pH levels in nematode-infested land; controlling other plant diseases, as well as insect and weed pests; using sanitation practices such as plowing, planting, and cultivating of nematode-infested fields only after working non-infested fields; cleaning equipment thoroughly with high pressure water or steam after working in infested fields; not using seed grown on infested land for planting non-infested fields unless the seed has been properly cleaned; rotating infested fields and alternating host crops with non-host crops; using nematicides; and planting resistant plant varieties.

Methods have been proposed for the genetic transformation of plants in order to confer increased resistance to plant parasitic nematodes. U.S. Pat. Nos. 5,589,622 and 5,824,876 are directed to the identification of plant genes expressed specifically in or adjacent to the feeding site of the plant after attachment by the nematode. The promoters of these plant target genes can then be used to direct the specific expression of detrimental proteins or enzymes, or the expression of antisense RNA to the target gene or to general cellular genes. The plant promoters may also be used to confer cyst nematode resistance specifically at the feeding site by transforming the plant with a construct comprising the promoter of the plant target gene linked to a gene whose product induces lethality in the nematode after ingestion. However, these patents do not provide any specific nematode genes that are useful for conferring resistance to nematode infection, and the methods are only useful for expressing genes specifically at the feeding sites for nematodes after attachment to the plant.

Recently, RNA interference (RNAi), also referred to as gene silencing, has been proposed as a method for controlling nematodes. When double-stranded RNA (dsRNA) corresponding essentially to the sequence of a target gene or mRNA is introduced into a cell, expression from the target gene is inhibited (See e.g., U.S. Pat. No. 6,506,559). U.S. Pat. No. 6,506,559 demonstrates the effectiveness of RNAi against known genes in Caenorhabditis elegans, but does not teach or suggest any novel genes that are essential for plant parasitic nematodes, and does not demonstrate the usefulness of RNAi for controlling plant parasitic nematodes.

A difficulty in finding agents that are active against nematodes, such as SCN, has been establishing an efficient, reproducible, and convenient bioassay method. The soybean cyst nematode can be propagated on normal soybean root explants. However, this technique requires the continual establishment of root explants because these organs have a determinant period of growth in culture. In contrast, soybean hairy roots generated by infecting soybean cotyledons with Agrobacterium rhizogenes exhibit indeterminate growth in tissue culture providing an alternative to normal root explants for monoxenic propagation and study of soybean cyst nematode (Cho et. al., (1998) Plant Sci. 138, 53-65). The A. rhizogenes can transfer the T-DNA of binary vectors in trans, thereby enabling the production of transgenic hairy roots containing foreign genes inserted in the T-DNA plasmid. This method has been used to produce transgenic roots in several plant species (Christey, (1997) Doran, P. M. (ed) Hairy roots: culture and application, Harwood, Amsterdam, pp. 99-111). The transgenic hairy roots can then be used to study the effect of transgene expression on any given phenotype.

There are difficulties in establishing root screening assays to determine efficacy of agents to control an obligate parasite such as SCN. Several weeks are required to establish a root culture, such as the hairy root culture. Only after the hairy root culture is established can soybean cyst nematode eggs or second stage juveniles (SCN J2) be inoculated. After inoculation of SCN J2, a month is required for the nematode to complete its life cycle. The known root assay which employs standard-size (approximately 9-10 cm in diameter), stand-alone Petri dishes is not amenable to high throughput assay. Moreover, because the root assay employs a plant host, Agrobacterium, and an animal parasite, significant variability in results can occur. For example, in a standard size Petri dish the roots are distributed over a large area making it more difficult for the nematode juveniles to locate a root, resulting in variable nematode distribution and infection rate and therefore more variable and less reliable assay results. Thus there is a need for an efficient, high throughput, and reproducible root bioassay that can be utilized with either normal root explants or hairy root explants, including, but not limited to, soybean explants.

### SUMMARY OF THE INVENTION

The present invention provides an improved method of screening for activity of a transgene of interest against a plant parasitic nematode using transgenic roots, which comprises use of multi-well cell culture plates instead of Petri dishes for inducing and analyzing parasitic nematode infection. The present method of invention utilizes multi-well plates thereby reducing the area of root distribution allowing for use of fewer nematodes for inoculation while showing improved likelihood of nematodes locating a root for attachment and feeding, thus resulting in an improved infection rate and more reliable, reproducible, and higher quality data than compared to the standard-size, stand-alone Petri dish assays. The multi-well plate assay further allows for economy of scale by using less media, less nematode inoculum and improved speed due to faster rate of full root infiltration per well and fewer plates to open and smaller areas to inoculate and analyze post infestation. The method of the invention is particularly useful for assaying transgene activity against soybean cyst nematode in soybean hairy roots.

### BRIEF DESCRIPTION OF THE DRAWINGS

FIG. 1 shows the sequence of the let-767 gene (SEQ ID NO.1)

FIG. 2 shows the sequence of the let-70 gene (SEQ ID NO:2).

FIG. 3 shows the sequence of the rpl-3 gene (SEQ ID NO. 3).

FIG. 4 shows the root-nematode infestation rate of various events of let-767 transformed roots in the multi-well assay plate.

FIG. 5 shows the root-nematode infestation rate of various events of let-70 transformed roots in the multi-well assay plate.

FIG. 6 shows the root-nematode infestation rate of additional various events of let-70 transformed roots in the multi-well assay plate.

FIG. 7 shows the root-nematode infestation rate of additional various events of rpl-3 transformed roots in the multi-well assay plate.

FIG. 8 compares the known Petri dish method of generating soybean hairy roots with the multi-well cell culture plate method of the present invention utilizing Williams 82 induced by the A. rhizogenes strain K599.

FIG. 9 compares the known Petri dish method of generating soybean hairy roots with the multi-well cell culture plate method of the present invention. Calculation of resources for each construct is based on assaying 20 events per construct with 8 replications per event, where each replicate is one Petri dish or one well in a multi-well plate.

### DETAILED DESCRIPTION OF THE INVENTION

The present invention may be understood more readily by reference to the following detailed description of the preferred embodiments of the invention and the examples included herein. Unless otherwise noted, the terms used herein are to be understood according to conventional usage by those of ordinary skill in the relevant art. In addition to the definitions of terms provided below, definitions of common terms in molecular biology may also be found in Rieger et al., 1991 Glossary of genetics: classical and molecular, 5^{th} Ed., Berlin: Springer-Verlag; and in Current Protocols in Molecular Biology, F.M. Ausubel et al., Eds., Current Protocols, a joint venture between Greene Publishing Associates, Inc. and John Wiley & Sons, Inc., (1998 Supplement). It is to be understood that as used in the specification and in the claims, “a” or “an” can mean one or more, depending upon the context in which it is used. Thus, for example, reference to “a cell” can mean that at least one cell can be utilized. It is to be understood that this invention is not limited to specific nucleic acids, specific cell types, specific host cells, specific conditions, or specific methods, etc., as such may, of course, vary, and the numerous modifications and variations therein will be apparent to those skilled in the art. It is also to be understood that the terminology used herein is for the purpose of describing specific embodiments only and is not intended to be limiting.

Throughout this application, various publications are referenced. The disclosures of all of these publications and those references cited within those publications in their entireties are hereby incorporated by reference into this application in order to more fully describe the state of the art to which this invention pertains. Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook and Russell, 2001 Molecular Cloning, Third Edition, Cold Spring Harbor Laboratory, Plainview, N.Y.; Sambrook et al., 1989 Molecular Cloning, Second Edition, Cold Spring Harbor Laboratory, Plainview, N.Y.; Maniatis et al., 1982 Molecular Cloning, Cold Spring Harbor Laboratory, Plainview, N.Y.; Wu (Ed.) 1993 Meth. Enzymol. 218, Part I; Wu (Ed.) 1979 Meth Enzymol. 68; Wu et al., (Eds.) 1983 Meth. Enzymol. 100 and 101; Grossman and Moldave (Eds.) 1980 Meth. Enzymol. 65; Miller (Ed.) 1972 Experiments in Molecular Genetics, Cold Spring Harbor Laboratory, Cold Spring Harbor, N.Y.; Old and Primrose, 1981 Principles of Gene Manipulation, University of California Press, Berkeley; Schleif and Wensink, 1982 Practical Methods in Molecular Biology; Glover (Ed.) 1985 DNA Cloning Vol. I and II, IRL Press, Oxford, UK; Hames and Higgins (Eds.) 1985 Nucleic Acid Hybridization, IRL Press, Oxford, UK; and Setlow and Hollaender 1979 Genetic Engineering: Principles and Methods, Vols. 1-4, Plenum Press, New York. Abbreviations and nomenclature, where employed, are deemed standard in the field and commonly used in professional journals such as those cited herein.

Also as used herein, the terms “nucleic acid” and “polynucleotide” refer to RNA or DNA that is linear or branched, single or double stranded, or a hybrid thereof. The term also encompasses RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made.

As used herein, the term “transgenic” refers to any plant, plant cell, callus, plant tissue, or plant part that contains all or part of at least one recombinant polynucleotide. In many cases, all or part of the recombinant polynucleotide is stably integrated into a chromosome or stable extra-chromosomal element, so that it is passed on to successive generations. For the purposes of the invention, the term “recombinant polynucleotide” refers to a polynucleotide that has been altered, rearranged, or modified by genetic engineering. Examples include any cloned polynucleotide, or polynucleotides, that are linked or joined to heterologous sequences. The term “recombinant” does not refer to alterations of polynucleotides that result from naturally occurring events, such as spontaneous mutations, or from non-spontaneous mutagenesis followed by selective breeding.

The present invention may be used to bioassay the efficacy of agents encoded by transgenes that are active against a variety of plant parasitic nematodes. The present invention may also be used to assay the ability of specific breeder selected lines, naturally occurring lines, or lines subjected to mutation-producing or -inducing agents, to resist a variety of plant parasitic nematodes. Some such plants and their pathogens are listed in Index of Plant Diseases in the United States (U.S. Dept. of Agriculture Handbook No. 165, 1960); Distribution of Plant-Parasitic Nematode Species in North America (Society of Nematologists, 1985); and Fungi on Plants and Plant Products in the United States (American Phytopathological Society, 1989). In a preferred embodiment, the present invention may be used to screen for agents that are detrimental to parasitic nematodes, and most particularly, to cyst nematodes, for example, Heterodera glycines, Heterodera shachtii, Heterodera avenae, Heterodera oryzae, Globodera pallida, Globodera rostochiensis, or Globodera tabacum.

One embodiment of the present invention employs recombinant expression vectors comprising a nucleic acid encoding an agent which is detrimental to a parasitic nematode, for example a dsRNA molecule or other polynucleotide, wherein expression of the vector in a host plant cell results in increased resistance to a parasitic nematode as compared to a wild-type variety of the host plant cell. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Another type of vector is a viral vector, wherein additional DNA segments can be ligated into the viral genome. Certain vectors are capable of autonomous replication in a host plant cell into which they are introduced. Other vectors are integrated into the genome of a host plant cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., potato virus X, tobacco rattle virus, and Geminivirus), which serve equivalent functions.

Recombinant expression vectors useful in the method of the invention comprise a nucleic acid of the invention in a form suitable for expression of the nucleic acid in a host plant cell, which means that the recombinant expression vector includes one or more regulatory sequences, selected on the basis of the host plant cells to be used for expression, which is operatively linked to the nucleic acid sequence to be expressed. With respect to a recombinant expression vector, “operatively linked” is intended to mean that the nucleotide sequence of interest is linked to the regulatory sequence(s) in a manner which allows for expression of the nucleotide sequence (e.g., in a host plant cell when the vector is introduced into the host plant cell). The term “regulatory sequence” is intended to include promoters, enhancers, and other expression control elements (e.g., polyadenylation signals). Such regulatory sequences are described, for example, in Goeddel, Gene Expression Technology: Methods in Enzymology 185, Academic Press, San Diego, Calif. (1990) and Gruber and Crosby, in: Methods in Plant Molecular Biology and Biotechnology, Eds. Glick and Thompson, Chapter 7, 89-108, CRC Press: Boca Raton, Fla., including the references therein. Regulatory sequences include those that direct constitutive expression of a nucleotide sequence in many types of host cells and those that direct expression of the nucleotide sequence only in certain host cells or under certain conditions. It will be appreciated by those skilled in the art that the design of the expression vector can depend on such factors as the choice of the host cell to be transformed, the level of expression of dsRNA or other polynucleotide desired, etc. The expression vectors of the invention can be introduced into plant host cells to thereby produce dsRNA molecules encoded by nucleic acids as described herein.

In one embodiment of the invention, the introduced polynucleotide may be maintained in plant cells of roots stably if it is incorporated into a non-chromosomal autonomous replicon or integrated into the plant chromosomes. Alternatively, the introduced polynucleotide may be present on an extra-chromosomal non-replicating vector and be transiently expressed or transiently active. Whether present in an extra-chromosomal non-replicating vector or a vector that is integrated into a chromosome, the polynucleotide preferably resides in a plant expression cassette. A plant expression cassette preferably contains regulatory sequences capable of driving gene expression in plant cells that are operatively linked so that each sequence can fulfill its function, for example, termination of transcription by polyadenylation signals. Preferred polyadenylation signals are those originating from Agrobacterium tumefaciens t-DNA such as the gene 3 known as octopine synthase of the Ti-plasmid pTiACH5 (Gielen et al., 1984, EMBO J. 3:835) or functional equivalents thereof, but also all other terminators functionally active in plants are suitable. As plant gene expression is very often not limited on transcriptional levels, a plant expression cassette preferably contains other operatively linked sequences like translational enhancers such as the overdrive-sequence containing the 5′-untranslated leader sequence from tobacco mosaic virus enhancing the polypeptide per RNA ratio (Gallie et al., 1987, Nucl. Acids Research 15:8693-8711). Examples of plant expression vectors include those detailed in: Becker, D. et al., 1992, New plant binary vectors with selectable markers located proximal to the left border, Plant Mol. Biol. 20:1195-1197; Bevan, M. W., 1984, Binary Agrobacterium vectors for plant transformation, Nucl. Acid. Res. 12:8711-8721; and Vectors for Gene Transfer in Higher Plants; in: Transgenic Plants, Vol. 1, Engineering and Utilization, eds.: Kung and R. Wu, Academic Press, 1993, S. 15-38.

Plant gene expression should be operatively linked to an appropriate promoter conferring gene expression in a temporal-preferred, spatial preferred, cell type-preferred, and/or tissue-preferred manner. Promoters useful in the expression cassettes of the invention include any promoter that is capable of initiating transcription in a plant cell present in the plant's roots. Such promoters include, but are not limited to those that can be obtained from plants, plant viruses and bacteria that contain genes that are expressed in plants, such as Agrobacterium and Rhizobium. Preferably, the expression cassette of the invention comprises a root-specific promoter or a parasitic nematode feeding cell-specific promoter.

In one embodiment of the present invention, the expression cassette comprises an expression control sequence operatively linked to a nucleotide sequence that is a template for the agent that is detrimental to the parasitic nematode. For example, when the detrimental agent is a dsRNA, the dsRNA template comprises (a) a first stand having a sequence substantially identical to from about 21 to about 400 or 500 consecutive nucleotides of an anti-nematode target gene; and (b) a second strand having a sequence substantially complementary to the first strand. In further embodiments, a promoter flanks either end of the template nucleotide sequence, wherein the promoters drive expression of each individual DNA strand, thereby generating two complementary RNAs that hybridize and form the dsRNA. In alternative embodiments, the nucleotide sequence is transcribed into both strands of the dsRNA on one transcription unit, wherein the sense strand is transcribed from the 5′ end of the transcription unit and the antisense strand is transcribed from the 3′ end, wherein the two strands are separated by 3 to 500 basepairs, and wherein after transcription, the RNA transcript folds on itself to form a hairpin.

One embodiment of the present invention allows screening for activity of a transgene of interest against a plant parasitic nematode. The steps of the method include: transforming an Agrobacterium with a binary vector comprising a nucleic acid encoding the transgene of interest and a selectable marker; wounding and inoculating a portion of a seedling of a plant with the transformed Agrobacterium; culturing the inoculated seedling portion on a medium which prevents growth of plant cells that do not contain said selectable marker gene, for a time sufficient to induce callus tissue; allowing the callus tissue to generate transgenic roots; inoculating the roots with a plant parasitic nematode; incubating the inoculated roots for a time sufficient to allow the nematode to complete it life cycle; and scoring the extent of nematode reproduction. In accordance with the method of the invention, the steps of inoculating roots with plant parasitic nematodes, incubating the inoculated roots with the nematodes, and scoring the extent of nematode infestation are performed in multi-well cell culture plates. The methods of scoring the extent of nematode reproduction include, but are not limited to, counting the number of cysts, egg masses, juvenile or adult nematodes, or galls and/or observing feeding sites and/or changes in the physiology or morphology of the roots. Other embodiments of the present invention include the screening of non-transgenic plant roots, such a breeder selected plants, natural variants within a populations or laboratory induced mutation populations that give may rise to plants having altered tolerance or resistance to nematodes.

The following examples are not intended to limit the scope of the claims to the invention, but are rather intended to be exemplary of certain embodiments. Any variations in the exemplified methods that occur to the skilled artisan are intended to fall within the scope of the present invention.

### EXAMPLE 1

### Binary Vector Construction for Soybean Transformation

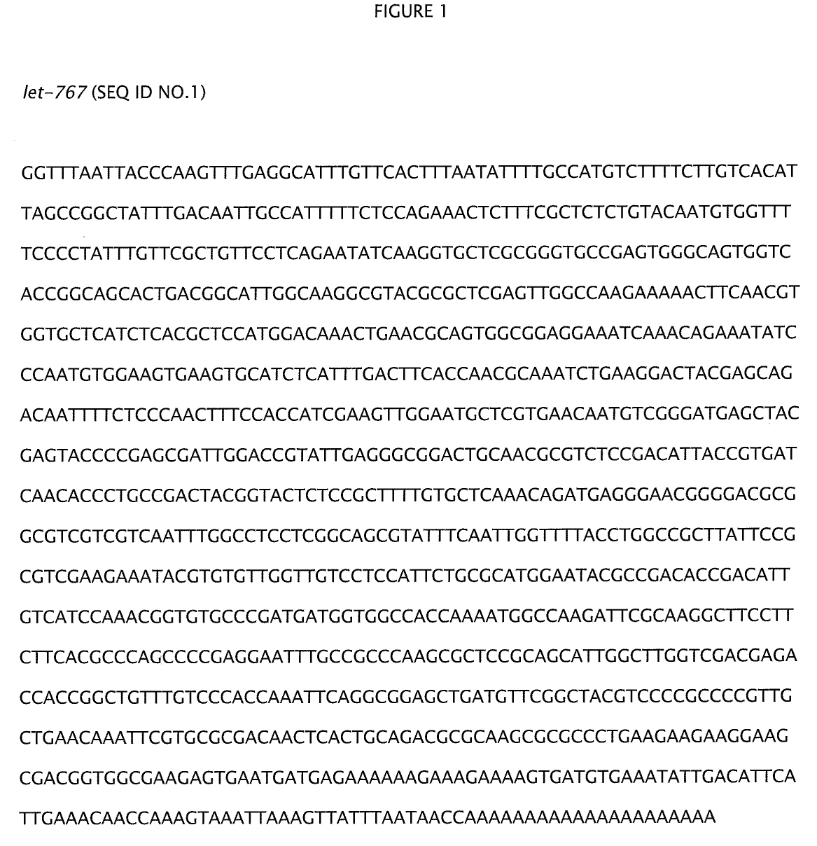

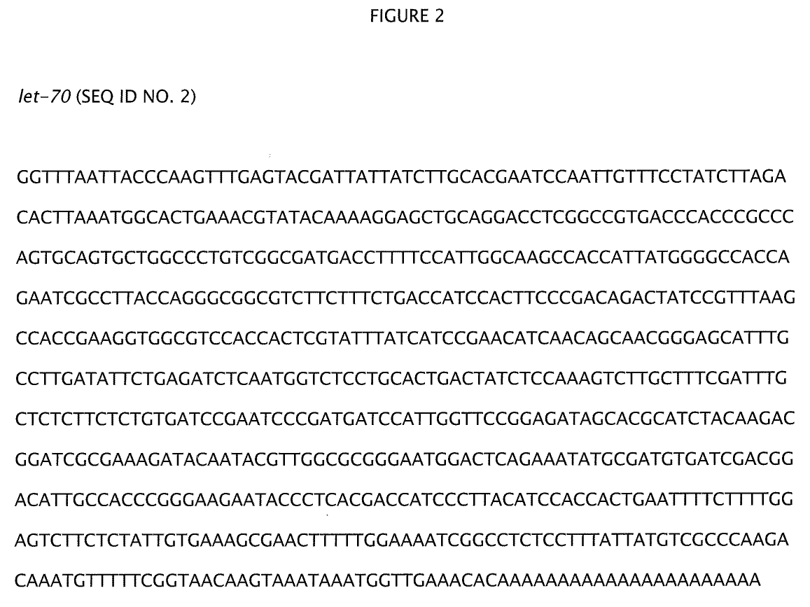

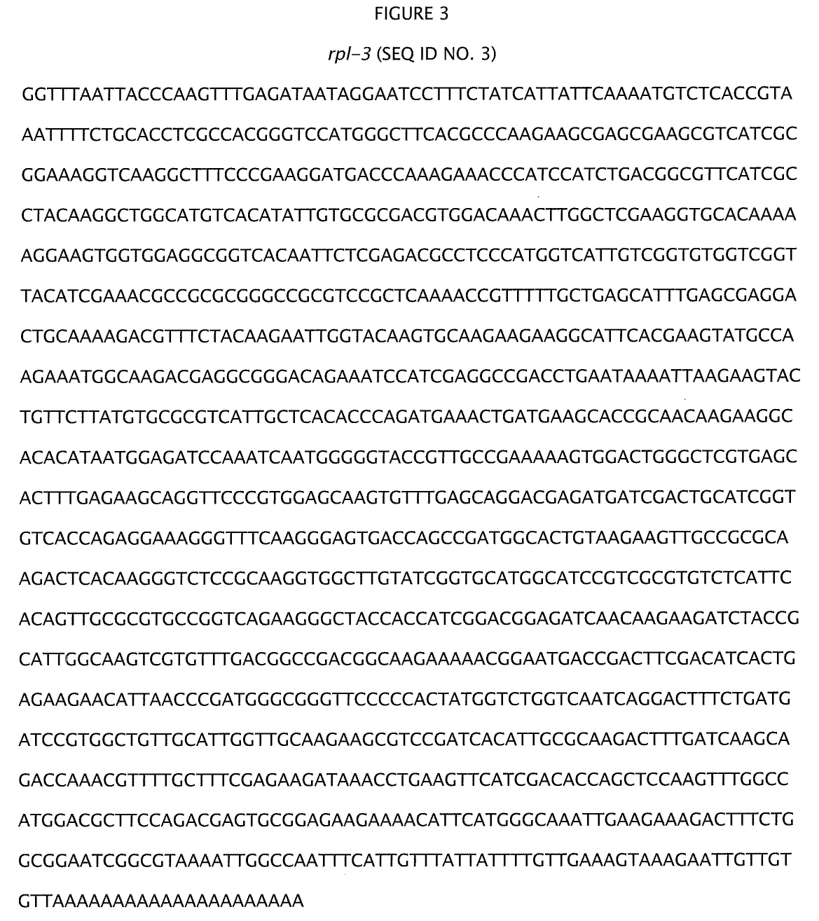

This exemplified method employs binary vectors containing a SCN target gene. The vectors consist of an antisense fragment of the target H. glycines let-767, let-70, or rpl-3 gene, a spacer, a sense fragment of the target geneand a vector backbone. The sequence of the let-767 gene (SEQ ID NO. 1) is set forth in FIG. 1, the sequence of the let-70 gene (SEQ ID NO.2) is set forth in FIG. 2, and the sequence of the rpl-3 gene (SEQ ID NO. 3) is set forth in FIG. 3. In these vectors, dsRNA for the target gene is expressed under a Super promoter (Ni, M. et al., Plant journal 7, 661-676, 1995). This promoter drives transgene expression at high level in many tissues including roots. The selection marker for transformation was a mutated AHAS gene from Arabidopsis thaliana that conferred resistance to the herbicide ARSENAL (imazepyr, BASF Corporation, Mount Olive, N.J.). The expression of mutated AHAS was driven by the Arabidopsis actin 2 promoter.

### EXAMPLE 2

### Generation of Transgenic Soybean Hairy-Root and Nematode Bioassay

In the present example, the transgenic hairy roots are used to study the effect of dsRNA generated from the binary vectors corresponding to the target genes in conferring cyst nematode resistance.

The binary vectors are transformed into A. rhizogenes K599 strain by electroporation (Cho et al., supra). The transformed strains of Agrobacterium are used to induce soybean hairy-root formation using the following protocol. Briefly, approximately five days before A. rhizogenes inoculation, seeds from soybean cultivar Williams 82 (SCN-susceptible) are sterilized with 10% bleach for 10 min and germinated on 1% agar at 25° C. with 16 hour/day lighting. Approximately three days before A. rhizogenes inoculation, a frozen stock of A. rhizogenes Strain K599 containing one of the binary vectors is streaked on LB+kanamycin (50 μg/ml) plates and incubated at 28° C. in darkness. Approximately one day before A. rhizogenes inoculation, a colony is picked from the plate and immersed into liquid LB+kanamycin (100 μg/ml). The culture is shaken at 28° C. for approximately 16 hours. The concentration of A. rhizogenes in the liquid culture is adjusted to OD₆₀₀=1.0. Cotyledons are excised from the soybean seedling and the adaxial side is wounded several times with a scalpel. 15 μl of A. rhizogenes suspension is inoculated onto the wounded surface, and the cotyledon is placed with the adaxial side up on a 1% agar plate for 3 days at 25° C. under 16 hour/day lighting. The cotyledons are then transferred onto MS plates containing 500 μg/ml Carbenicillin (to suppress A. rhizogenes) and 0.5 μM imazepyr. After culturing the cotyledons on selection media for 2 weeks, hairy roots are induced from the wounding site. The roots resistant to imazepyr and growing on the selection media are harvested and transferred onto fresh selection media of the same composition and incubated at 25° C. in darkness. Two weeks after harvesting hairy roots and culturing them on selection media, the hairy roots are subcultured onto MS media containing Carbenicillin 500 μg/ml but not imazepyr in multi-well plates, in particular for this example 6-well plates were utilized. Non-transgenic hairy roots from soybean cultivar Williams 82 (SCN susceptible) and Jack (SCN resistant) are also generated by using non-transformed A. rhizogenes, to serve as controls for nematode growth in the assay.

A bioassay to assess nematode resistance is performed on the transgenic hairy-root transformed with the vectors and on non-transgenic hairy roots from Williams 82 and Jack as controls. Hairy root cultures of each line that occupy at least half of the well are inoculated with surface-decontaminated race 3 of soybean cyst nematode (SCN) second stage juveniles (J2) at the level of 500 J2/well. The plates are then sealed and put back into the incubator at 25° C. in darkness. Several independent hairy root lines are generated from each binary vector transformation and the lines used for bioassay. As an example of the nomenclature used for the transgenic let-767 line, Let-767 02 indicates Line 2 generated from transformation with the let-767 vector. Four weeks after nematode inoculation, the cyst number in each well is counted.

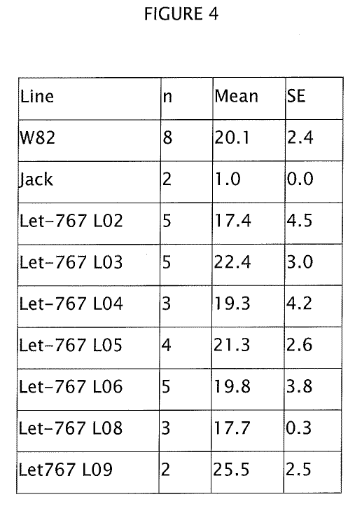

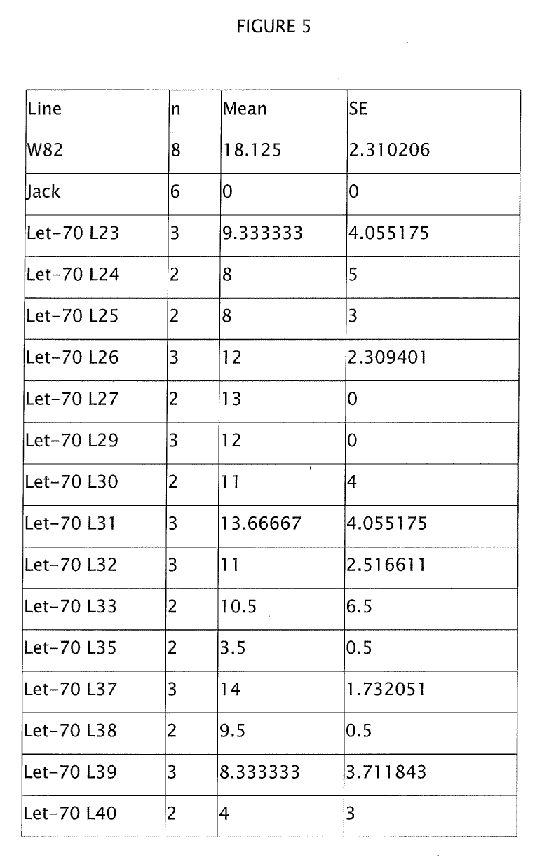

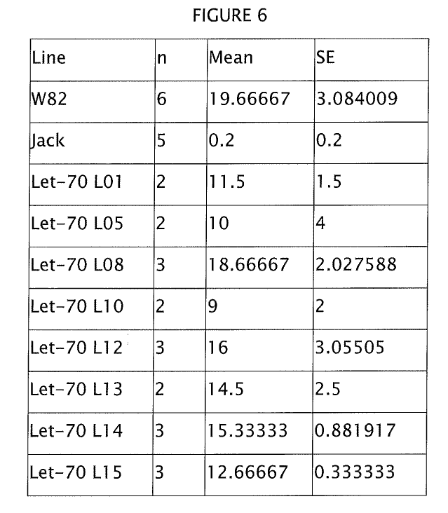

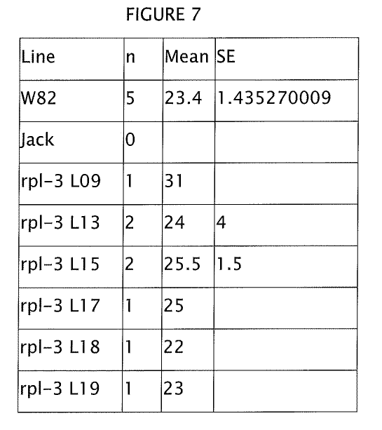

For each transformation line, several replicated wells (number of replicates indicated by “n”,) the average number of cysts per well (MEAN), and the standard error (SE) values are determined. The multi-well results for let-767 are found in FIG. 4, let-70 results are found in FIG. 5 and FIG. 6, and rpl-3 results are found in FIG. 7. The results for these multi-well assays show that the Means between events of a particular vector are consistent with a low standard error value. Further these result demonstrate that the the multi-well assay is capable of discerning between more resistant events, such as the let-70 lines with Mean cyst counts of about 4-14 in FIG. 5 and 9-19 in FIG. 6, and less resistant events such as let-767, with Mean cyst count values of about 17-26 in FIG. 4, and rpl-3, with Mean cyst count values of about 22-31 in FIG. 7.

### EXAMPLE 3

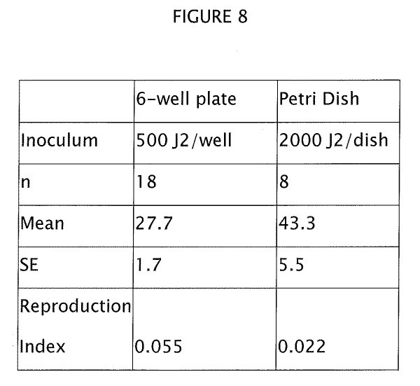

Comparison of multiple transgenic soybean hairy-root events utilizing the multi-well nematode bioassay of the present invention and the single Petri dish assay of the prior art. A comparison of the Petri dish method of the prior art and the multi-well plate of the present invention was conducted following the same, or substantially the same methods outlined in Example 2. The media was dispensed into either a Petri dish or a well of a six-well multi-well plate and the Williams 82 roots subcultured into the wells were induced by the Agrobacterium rhigenes strain K599 and allowed to grow as described in Example 2. The wells were inoculated with either 500 J2/well for the six-well plate or 2000 J2/dish for the Petri dishes. Four weeks following inoculation the number of cysts were counted/well or plate (Mean), the standard error (SE) value calculated and the reproduction index (final cyst number/number of J2 inoculum) calculated. The results (see FIG. 8) show that the reproduction index is over two-times higher for the multi-well assay as compared to the Petri dish assay demonstrating an increased chance of nematodes infecting the roots thus requiring less inoculum. This also results in a more uniform infection and less variation in nematode development.

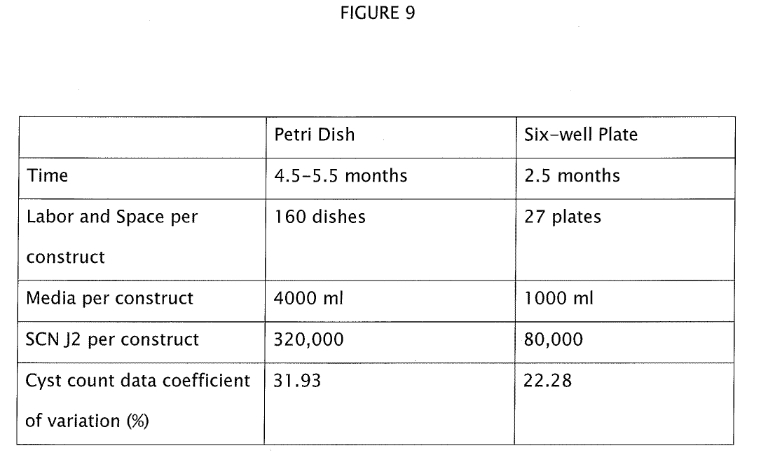

Following the same, or substantially the same, protocols outlined in Example 1 and Example 2, multiple vectors for various target genes were created and tested utilizing the nematode root-assay. Certain of the assays were performed in Petri dishes and certain assays were performed in multi-well plates. By analyzing this large data set of the Petri dishes together and the multi-well (six-well in this experiments) plates together, it is possible to demonstrate the improved consistency of data in the multi-well plate (coefficient of variation of 22.28% based on data from over 300 wells) over that of the single well Petri-dish of the prior art (coefficient of variation of 31.93% based on data from over 1000 dishes) (see FIG. 9). This experiment also demonstrates the improved speed (2.5 months vs. approximately 5 months), reduced amount of media used, and greatly reduced amount of J2 inoculum required for the multi-well plate compared to the Petri-dish—a 4 fold reduction.

Those skilled in the art will recognize, or will be able to ascertain using no more than routine experimentation, many equivalents to the specific embodiments of the invention described herein. Such equivalents are intended to be encompassed by the following claims.

What is claimed is:

1. An improved method of screening for activity of a transgene of interest against a plant parasitic nematode which comprises the steps of a) transforming an Agrobacterium with a vector comprising a nucleic acid encoding the transgene of interest and a selectable marker; b) inoculating a portion of a seedling of a plant with the transformed Agrobacterium; c) culturing the inoculated seedling portion on a medium which prevents growth of plant cells that do not contain said selectable marker gene, for a time sufficient to induce callus tissue; d) culturing the callus containing the selectable marker gene and the transgene of interest; e) inducing the callus tissue to produce roots; f) inoculating the roots with a plant parasitic nematode; g) incubating the inoculated roots for a time sufficient to allow the nematode to complete its life cycle; and h) scoring the extent of nematode reproduction; the improvement comprising performing steps f) through h) in multi-well plates.

2. The method of claim 1, wherein the plant is selected from the group consisting of corn, potato, cotton, sugar beet, and soybean.

3. The method of claim 1, wherein the plant is a soybean.

4. The method of claim 3, wherein the nematode is soybean cyst nematode.

5. The method of claim 1, wherein the nematode is root-knot nematode.

6. The method of claim 1, wherein the nematode is a cyst-type or a lesion-type.

7. The method of claim 1, wherein the Agrobacterium is Agrobacterium rhizogenes or Agrobacterium tumefaciens.

8. The method of claim 1, wherein the roots are hairy roots or normal roots.

9. An improved method of screening for resistance of a plant against a plant parasitic nematode which comprises the steps of a) transforming an Agrobacterium with a vector comprising a selectable marker; b) inoculating a portion of a seedling of the plant with the transformed Agrobacterium; c) culturing the inoculated seedling portion on a medium which prevents growth of plant cells that do not contain said selectable marker gene, for a time sufficient to induce callus tissue; d) culturing the callus containing the selectable marker gene; e) inducing the callus tissue to produce roots; f) inoculating the roots with a plant parasitic nematode; g) incubating the inoculated roots for a time sufficient to allow the nematode to complete its life cycle; and h) scoring the extent of nematode reproduction; the improvement comprising performing steps f) through h) in multi-well plates.

10. The method of claim 9, wherein the plant is selected from the group consisting of corn, potato, cotton, sugar beet, and soybean.

11. The method of claim 9, wherein the plant is a soybean.

12. The method of claim 11, wherein the nematode is soybean cyst nematode.

13. The method of claim 9, wherein the nematode is root-knot nematode.

14. The method of claim 9, wherein the nematode is a cyst-type or a lesion-type.

15. The method of claim 9, wherein the Agrobacterium is Agrobacterium rhizogenes or Agrobacterium tumefaciens.

16. The method of claim 9, wherein the roots are hairy roots or normal roots.
